(19) United States
(12) Patent Application Publication
(10) Pub. No.: US 2023/0055392 A1
(43) Pub. Date: Feb. 23, 2023

(54) IMPLANTABLE MEDICAL DEVICE WITH A WAKE-UP DEVICE

(71) Applicant: BIOTRONIK SE & Co. KG, Berlin (DE)

(72) Inventors: Christian Moss, Berlin (DE); André Seidelt, Berlin (DE); Andreas Arndt, Berlin (DE); Olaf Skerl, Bad Doberan (DE); Thomas Finnberg, Barsbuettel (DE)

(73) Assignee: BIOTRONIK SE & Co. KG, Berlin (DE)

(21) Appl. No.: 17/914,858

(22) PCT Filed: Mar. 24, 2021
(86) PCT No.: PCT/EP2021/057528

§ 371 Date: Sep. 27, 2022

(30) Foreign Application Priority Data
Mar. 30, 2020 (EP) 20166541.1

(51) Int. Cl. G04G 3/02; G04D 7/00

(52) U.S. Cl. CPC G04G 3/02; G04D 7/003; G04F 5/06

(57) ABSTRACT

An implantable medical device comprises an electronic functional device for performing a function of said implantable medical device, said electronic functional device having an operational state for performing said function and a switched-off state. A wake-up device serves for transferring said functional device from said switched-off state to said operational state. The wake-up device comprises a first timer circuit for repeatedly transferring the functional device to the operational state according to a predetermined first timing scheme, a detection device for detecting a signal from a signal source external to the implantable medical device, and a second timer circuit for repeatedly switching the detection device to a reception state according to a second timing scheme.

### CROSS-REFERENCE TO RELATED APPLICATIONS

This application is the United States National Phase under 35 U.S.C. § 371 of PCT International Patent Application No. PCT/EP2021/057528, filed on Mar. 24, 2021, which claims the benefit of European Patent Application No. 20166541.1, filed on Mar. 30, 2020, the disclosures of which are hereby incorporated by reference herein in their entireties.

### TECHNICAL FIELD

The present invention concerns an implantable medical device according to the preamble of claim 1.

### BACKGROUND

Such implantable medical device comprises a functional device in the shape or form of an electronic module or electric circuit for performing a function of the implantable medical device, the functional device having an operational state for performing the function and a switched-off state. A wake-up device serves to transfer the functional device from the switched-off state to the operational state in order to execute the predetermined function in the operational state.

Such an implantable medical device may be a measurement system that can be implanted in a patient's vessel, for example, to measure a parameter such as a blood pressure or a blood flow within the vessel. The measurement system can be used to monitor a patient's condition, for example, for observing and diagnosing a course of disease, wherein the implantable medical device may be designed to communicate with an external device (located outside the patient) to transmit the measurement results to the external device after measurement by the implantable medical device.

Alternatively, such an implantable medical device may be designed as a stimulation device, for example, with a pacemaker or neurostimulation function.

An implantable medical device comprises a functional device in the form of an electronic module or electric circuit, which is formed, for example, by a processor and serves to perform a function in an active operational state of the medical device, for example, a measurement function to measure a parameter of a patient, for example, a blood pressure. To operate the functional device, the implantable medical device comprises an energy storage device, particularly in the form of an electric battery, which feeds the functional device and supplies it with power in its operational state. Such an implantable medical device should usually remain in a patient after implantation for a long period of time, for example, several years, which requires that the energy storage has a corresponding capacity to supply the functional equipment. However, because such an implantable medical device should, for example, have a small shape in order to be able to implant the medical device into small blood vessels, for example, an artery or the like, the available space for the energy storage device and thus also the capacity of a battery realizing the energy storage device is limited.

From the desire to miniaturize a battery for use in small implantable medical devices it follows that the power consumption of the medical device should be low. For this it is desirable that the medical device is not permanently active, but is only switched to an active operational state (in which the functional device can perform a predetermined function, for example, to measure a patient's parameter) if required, but otherwise the implantable medical device is largely inactive and therefore consumes no or at least only very little power in passive phases. For this purpose, the functional device of the implantable medical device is preferably in the switched-off state for most of the time and can be transferred from the switched-off state to the operational state by the wake-up device in order to then carry out a predetermined function in the operational state. After executing the function, the functional device switches back to the switched-off state until the functional device is woken up again by the wake-up device and thus is transferred to the operational state.

Herein the wish exists to be able to wake up the functional device flexibly in order to be able to switch on the functional device, for example, in a user-triggered manner on the one hand, but on the other hand also to enable a user-independent switching of the functional device to the operational state. This should come at a low power consumption.

An implantable neurostimulation system known from U.S. Publication No. 2018/0161576 A1 comprises a GMR sensor designed to detect the presence of a magnetic field. By detecting a magnetic field, the system can be switched on so that a stimulation function can be performed.

The present disclosure is directed toward overcoming one or more of the above-mentioned problems, though not necessarily limited to embodiments that do.

### SUMMARY

It is an object of the instant invention to provide an implantable medical device that enables energy-efficient operation and may in this way remain implanted in a patient over a prolonged period of time.

At least this object is achieved by an implantable medical device comprising the features of claim 1.

Accordingly, the wake-up device comprises a first timer circuit for repeatedly transferring the functional device into the operational state according to a first timing scheme, a detection device for detecting a signal from a signal source external to the implantable medical device, and a second timer circuit for repeatedly transferring the detection device into a reception state according to a second timing scheme.

By using the wake-up device, the functional device of the implantable medical device may be in a switched-off state over prolonged phases and may be switched on as needed to then perform a predetermined function, such as a measurement or the like, in an operational state. The implantable medical device may have a low energy consumption when the functional device is switched off, and may be transferred to the operational state, in which an intended function can be performed, by switching on the functional device. Because by using the wake-up device the implantable medical device may be switched on only when needed, a low overall energy consumption may be achieved, such that even with a small form factor of an energy storage device, for example, in the shape of a battery, the implantable medical device may be functional in an implanted condition within a patient over a long period of time, for example, several years.

The wake-up device comprises different timer circuits. A first timer circuit is used to repeatedly transfer the functional device to the operational state according to a given first timing scheme. The first timer circuit is thus used to switch on the functional device according to a predetermined timing scheme independently of the detection of a signal of an external signal source.

For example, the first timer circuit may periodically switch on the functional device so that the functional device is caused to transfer from the switched-off state to the operational state via the first timer circuit at regular intervals and can thus perform a predetermined function.

For example, the first timer circuit may be designed to switch on the functional device at regular intervals between a few hours and a few days. For example, the first timer circuit may be designed to switch on the functional device twice a day, for example, at a predetermined time in the morning and at a predetermined time in the evening. The functional device may then, in one embodiment, remain in the operational state for a predetermined period of time, for example, a few microseconds, a few milliseconds, a few seconds or few minutes, to perform the predetermined intended function, for example, a measurement of the patient's blood pressure or another parameter, and may switch back to the switched-off state after the function has been performed.

The first timer circuit may cause the functional device to switch on periodically. However, this is not mandatory. A given first timing scheme may also provide for irregular intervals for the repeated switching on of the functional device, i.e., for the transfer of the functional device into the operational state.

In one embodiment, the first timer circuit comprises an oscillator circuit adapted to generate a clock signal for determining a time to transfer the functional device to the operational state. The oscillator circuit thus generates a specific clock, which is used to determine whether a point in time for switching on the functional device has been reached. If the clock signal of the oscillator circuit indicates, for example, that a predetermined period of time has lapsed, an actuating signal is generated by means of which, for example, a switch for switching on the functional device and thus for energizing the functional device for carrying out a specified function is actuated.

The oscillator circuit may, for example, comprise a so-called RC oscillator. Such an RC oscillator is implemented with RC elements (i.e., a combination of an electrical resistor and a capacitor) which generate sinusoidal oscillations and thus realize an oscillator. Such an RC oscillator may be designed simply and cost-effectively and may generate a clock signal in an energy-efficient manner, i.e., with low power consumption.

For example, if an RC oscillator is used in the oscillator circuit of the first timer circuit, such an RC oscillator may have comparatively low accuracy. Energy-efficient operation is thus made possible potentially at the expense of accuracy. In order to enable an accurate timing, the first timer circuit may, for example, comprise a calibration oscillator—in addition to the oscillator circuit—which serves to calibrate the oscillator circuit and is formed, for example, by a quartz oscillator. Calibration may, for example, be performed repeatedly while the oscillator circuit is operating, for example, by turning on the calibration oscillator at regular or irregular intervals for generating a clock signal parallel to the clock signal of the oscillator circuit for a predetermined period of time to calibrate the clock signal of the oscillator circuit according to the clock signal of the calibration oscillator.

For example, the calibration oscillator may be turned on at time intervals between 100 seconds and 10,000 seconds, for example, between 500 seconds and 5000 seconds, for example, after every 1024 seconds, to then operate in parallel with the oscillator circuit for a predetermined calibration period, for example, between 10 seconds and 100 seconds, for example, 50 seconds, and thus generate a clock signal for calibration according to which the clock signal of the oscillator circuit may be calibrated.

The calibration oscillator may operate with significantly greater accuracy than the oscillator circuit and thus may enable calibration. In a calibration phase, the clock signal of the oscillator circuit is calibrated using the calibration oscillator. By switching on the calibration oscillator (repeatedly) for a comparatively short period of time for calibration, a high degree of accuracy may be achieved in the clock signal of the oscillator circuit, while achieving an overall rather low power consumption of the first timer circuit.

The second timer circuit is used to switch the detection device to a reception state in which the detection device may receive a signal from an external signal source. By means of the second timer circuit, the detection device may be switched to the reception state at regular or irregular intervals so that the detection device may detect a signal from the external signal source.

In one embodiment, the second timer circuit is, for example, configured to periodically switch the detection device to the reception state for a predetermined reception period. For example, the detection device may be switched on at a periodic, regular time interval between 1 second and 10 seconds, for example, between 3 seconds and 8 seconds, for example, 4 seconds, to then remain in the reception state for a predetermined period, for example, between 50 μs and 1000 μs, for example, between 100 μs and 400 μs, for example, for 200 μs.

Alternatively, the detection device may be switched on at irregular intervals according to a predetermined (second) timing scheme.

For example, the detection device comprises a detection sensor which may be used to detect a signal from an external signal source. The detection sensor may, for example, be a sensor by means of which the presence of an external magnetic field may be detected. Such a sensor may, for example, be implemented by a so-called GMR sensor (GMR stands for Giant Magnetoresistance), in which a change in resistance occurs when a magnetic field is present, so that a sensor signal may be generated from the change in resistance.

Generally, the detection device is configured to generate a signal that depends on the detection of a signal from an external signal source. The signal source may, for example, be a permanent magnet or an electromagnet. The signal source thus generates a magnetic field whose presence may be detected by the detection device in order to generate a sensor signal that depends on the presence of the external magnetic field.

By means of the signal of the detection device, the functional device is caused to be switched from the switched-off state to the operational state if a signal of an external signal source is detected. In one embodiment, the wake-up device may comprise an inversion circuit configured to invert the signal generated by the detection device and to output an inverted signal obtained by the inversion circuit.

For example, the inversion circuit may be connected to the first timer circuit so that the inversion circuit outputs the inverted signal to the first timer circuit, which is thus controlled to actuate a switch to switch on the system of the functional device and thus to transfer the functional device to the operational state. In this embodiment, hence, the detection device is connected to the first timer circuit via the inversion circuit, so that the functional device is switched via the first timer circuit.

The inversion circuit is configured to invert the signal of the detection device. The inversion circuit inverts a high signal level (logical High) into a low signal level (logical Low) and vice versa. If, for example, the signal of the detection device exhibits a low signal level on occurrence of an external magnetic field, the inversion circuit inverts the signal into a high signal level which may be supplied as a pulse to the first timer circuit in order to switch on the functional device.

In one embodiment, the functional device remains in the operational state for a predetermined period of time after it has been transferred to the operational state in order to carry out its predefined function, and then reverts back to the switched-off state. For example, the duration may be a few seconds or a few minutes. For example, a measurement may be carried out in the operational state, for example, a blood pressure measurement, wherein the functional device reverts to the switched-off state after conclusion of the measurement, and the implantable medical device thus switches back to a low-energy state.

In the operational state, for example, a parameter such as pressure, flow, temperature, etc. may be measured. In addition or alternatively, emission of stimulation energy may take place, for example, within the framework of a pacemaker function or within the framework of a neurostimulation function.

In one embodiment, the implantable medical device comprises a volatile memory, but no non-volatile memory. Measured values obtained during a measurement are temporarily stored in the volatile memory (RAM) and are transferred to an external device during a phase of the operational state, so that a measurement takes place during the phase of the operational state and measurement results are transferred with a minimum time delay to an external device, for example, a monitoring device external to the patient. Measured values are thus stored (only) temporarily within the medical device during a phase of the operational state, but are not retained in the memory between successive phases of operational states.

In another embodiment, the implantable medical device comprises a non-volatile or permanent memory, wherein the non-volatile memory may be arranged within, connected to and/or powered by the wake-up device, particularly arranged within, connected to and/or powered by the first timer circuit or the second timer circuit. Preferably, the non-volatile memory be accessed by the functional device in order to save a status information about the last activation until the next activation.

In one embodiment, the first timer circuit and/or the second timer circuit comprise at least one electronic component which is implemented in a sub-threshold technology. The first timer circuit and the second timer circuit may, for example, each be realized by their own electronic chip. The respective chip may be implemented in a sub-threshold technology. Chips implemented in sub-threshold technology may provide for an electronic circuit with low power consumption. The first timer circuit and the second timer circuit may thus have a comparatively low energy consumption, so that the operation of the first timer circuit and the second timer circuit, which operate continuously even when the functional device is switched off, may therefore be energy-efficient.

The sub-threshold technology is a chip technology in which transistors designed as MOSFETs are operated in integrated circuits in the so-called sub-threshold region. This mode of operation enables low power consumption.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea(s) behind the present invention shall subsequently be explained in more detail by referring to the embodiments shown in the figures. Herein:

FIG. 1 shows a schematic view of a medical device implanted in a patient, along with an external signal source and an external monitoring device;

FIG. 2 shows a schematic view of an implantable medical device;

FIG. 3 shows a schematic view of a wake-up device of the medical device;

FIG. 4 shows a functional view of the wake-up device;

FIG. 5 shows a view of the medical device together with an external signal source;

FIG. 6 shows a graphical view of the magnetic flux density of the external signal source as a function of the distance from the external signal source; and

FIG. 7 shows a view of the time dependent current of the medical device in dependence on the switching on of a detection device and the calibration of a first timer circuit.

### DETAILED DESCRIPTION

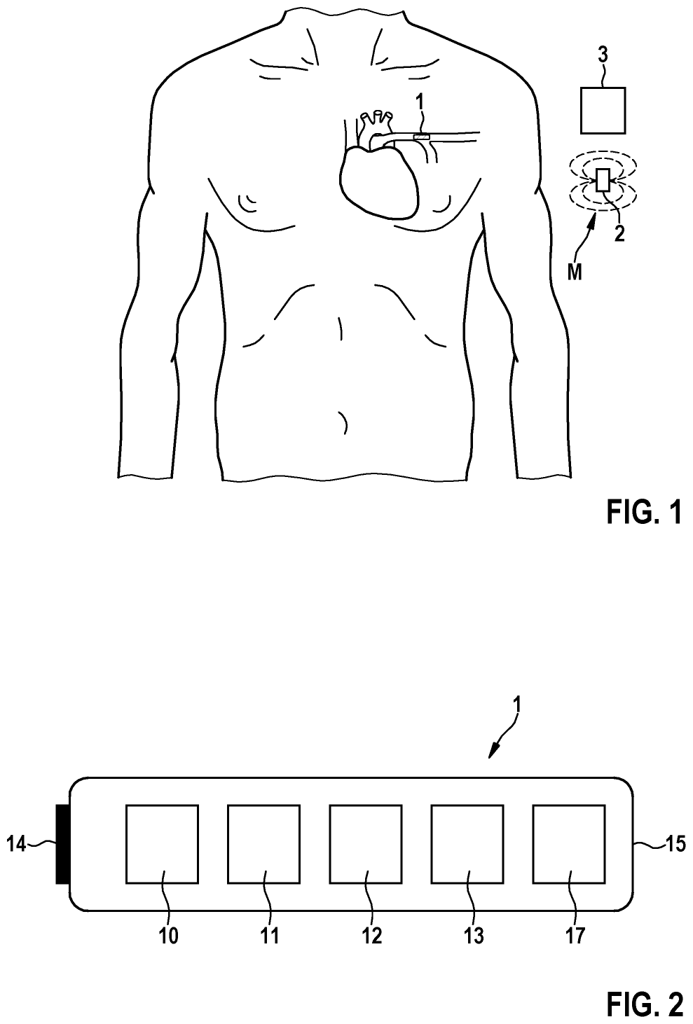

FIG. 1 shows a schematic view of a medical device 1, which is configured as a measuring system for measuring a parameter of a patient, for example, blood pressure, and is implanted in a vessel, for example, an artery, of the patient.

The medical device 1 is used to perform a function in a patient over a prolonged period of time, such as a measurement function or a cardiac or neuronal stimulation function for the purpose of therapy. For example, the medical device 1 shall remain in a patient for multiple years in order to record measurement data during the lifetime of the medical device 1 and to communicate with an external device 3, so that the measurement data may be used to diagnose or monitor the condition of the patient.

Such a medical device 1 should be small in size. As schematically shown in FIG. 2, the medical device 1, for example, comprises a housing 15, which, in one embodiment, may comprise an essentially cylindrical housing shape, with a length between 10 mm and 40 mm and a diameter, for example, between 3 mm and 10 mm, wherein other dimensions are also conceivable and possible.

Such a medical device 1 comprises, in the example of the shown embodiment, an electronic functional device 10 which is formed, for example, by a processor and serves to perform a predetermined function, for example, a measuring function or a therapy function. The medical device 1 in addition comprises a volatile memory 11 in the form of a RAM (Random Access Memory), a wake-up device 12, an energy storage device 13, for example, in the form of a battery, and a communication device 17 for communicating with an external device 3. The different functional units are encapsulated together in the housing 15 in a fluid-tight manner.

The medical device 1 in addition comprises, for example, a measurement sensor 14, which is used together with the functional device 10 to perform a measurement in order to record measurement data, for example, to measure a pressure within a patient's vessel. A measurement may be performed over a predetermined period of time, for example, a few seconds or a few minutes, with measurement data being stored temporarily in the memory 11 during a measurement and communicated to the external device 3 via the communication device 17. Because the memory 11 is configured as a volatile memory, measurement data is not stored permanently, but is transmitted immediately (with only a minimum delay due to internal processing) to the external device 3 via the communication device 17 during a measurement.

Because the medical device 1 has small dimensions, the size of the energy storage device 13 is also necessarily limited. Because the medical device 1 is to remain in a patient and be operative for a prolonged period of time, for example, several years, it is desired that the medical device 1 operates energy-efficiently, thus requiring little power, but still functions reliably to perform one or more predetermined functions.

In order to reduce the energy consumption of the medical device 1, in the embodiment of FIG. 2 the functional device 10 does not operate continuously and at all times, but is only switched from a switched-off state to an operational state when required in order to carry out a function in the operational state. In the switched-off state the functional device 10 is shut down and causes no or only a very limited power consumption, so that in the switched-off state of the functional device 10 the system of the medical device 1 exhibits a low overall power consumption.

In order to transfer the functional device 10 from the switched-off state to the operational state, the wake-up device 12 is provided, which serves to switch on the functional device 10 on the one hand independently of an external trigger and on the other hand depending on an external signal source 2 (see FIG. 1).

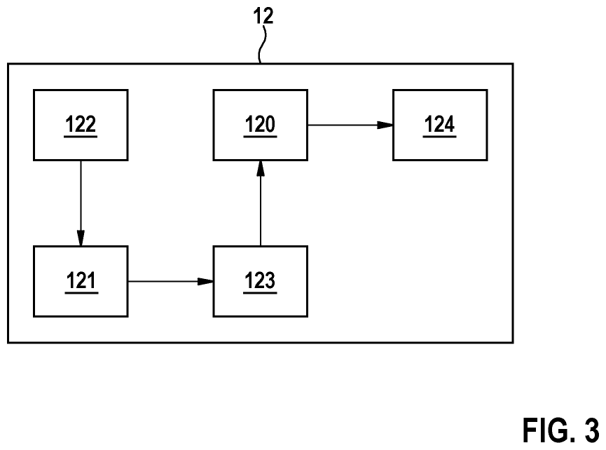

Referring now to FIG. 3, the wake-up device 12 comprises a first signal source 120 which serves to periodically control a switching device 124 for switching on the functional device 10 in order to transfer the functional device 10 at regular intervals from the switched-off state to the operational state by means of the switching device 124. The first timer circuit 120 is hence used to cause the functional device 10 to be switched on at regular intervals independently of an external signal source 2, for example, to carry out measurements at regular intervals, for example, several times a day or several times per hour.

The wake-up device 12 in addition comprises a second timer circuit 122 which is used to control a detection device 121. By means of the second timer circuit 122, the detection device 121 is switched on at a comparatively large clock rate in order to detect a signal from an external signal source 2. A signal from the detection device 121 is supplied via an inversion circuit 123 to the first timer circuit 120, so that the first timer circuit 120 may drive the switching device 124 to switch on the functional device 10 when a signal from an external signal source 2 is detected via the detection device 121.

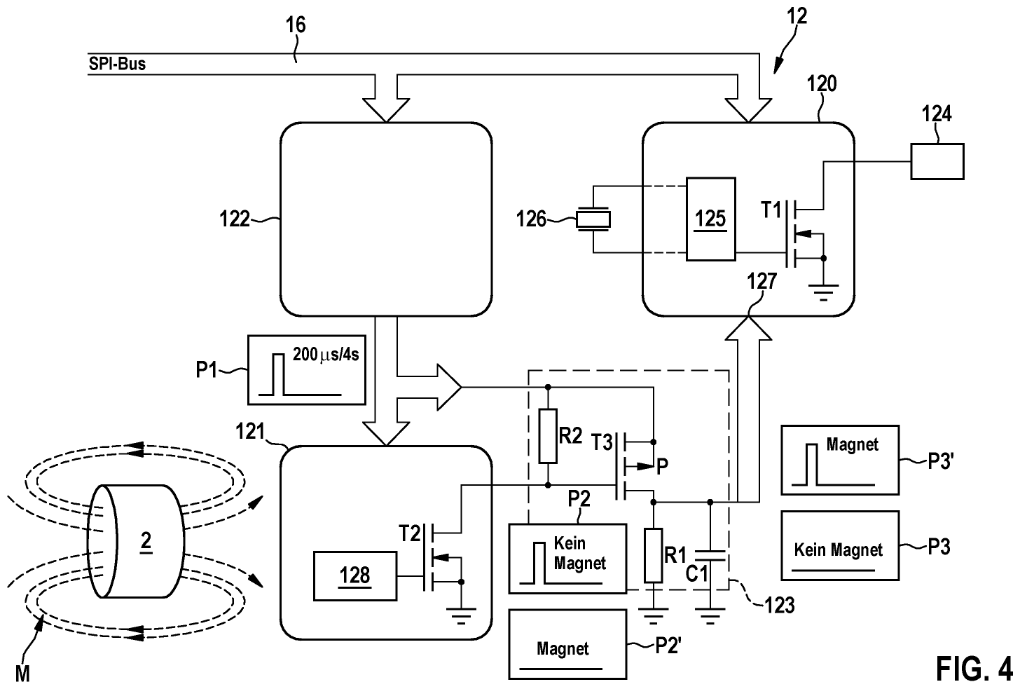

FIG. 4 illustrates in a functional drawing the function of the wake-up device 12.

The first timer circuit 120 comprises an oscillator circuit 125, which, for example, comprises an RC oscillator for generating a clock signal. By means of the oscillator circuit 125, a control signal is generated which causes the switching device 124 to be actuated via a transistor T1 to switch on the functional device 10.

For example, the oscillator circuit 125 may be configured to determine a time that has elapsed after a previous measurement based on the clock signal generated. In this way, the oscillator circuit 125 may actuate the functional device 10, for example, at regular intervals, for example, every 6, every 12 or every 24 hours. Alternatively, a specific time point may be determined by means of the oscillator circuit 125, so that the functional device 10 is transferred into the operational state on the basis of the control signal generated by the oscillator circuit 125 at predetermined times.

The oscillator circuit 125 uses, for example, an RC oscillator to generate the clock signal. Such an RC oscillator may be energy efficient, but usually has a comparatively low accuracy. For this reason, in addition to the oscillator circuit 125, the first timer circuit 120 comprises a calibration oscillator 126, which, for example, is formed by a quartz oscillator and serves to calibrate the RC oscillator of the oscillator circuit 125.

For example, the quartz oscillator of the calibration oscillator 126 may be turned on at regular intervals to produce a calibrating clock signal that may be used to calibrate the clock signal of the RC oscillator of the oscillator circuit 125. For example, the calibration oscillator 126 may be turned on periodically at a time interval between 100 seconds and 10,000 seconds, for example, every 1024 seconds, to generate a calibrating clock signal to calibrate the RC oscillator over a predetermined period of time, for example, 50 seconds.

The calibration oscillator 126 may have a high accuracy. Because the calibration oscillator 126 does not work continuously, the first timer circuit 120 may operate energy-efficiently at a low power consumption.

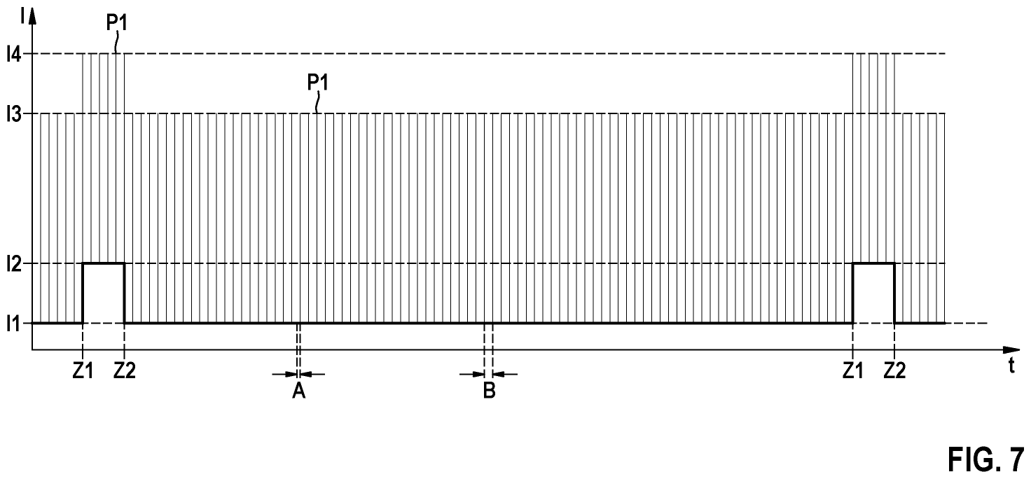

The second timer circuit 122 serves to control the detection device 121. The second timer circuit 122 serves to, for example, periodically actuate the detection device 121 and for this generates, at a regular pulse interval B (see FIG. 7), a signal P1 having a predetermined pulse duration A and serving to switch on the detection device 121. For example, the pulse interval B may lie between 1 second and 10 seconds, for example, 4 seconds. The pulse duration A may, for example, lie between 50 μs and 1000 μs, for example, between 100 μs and 400 μs, for example, 200 μs.

The detection device 121 is switched on for the duration of a pulse, i.e., for a time period corresponding to the pulse duration A, and is thus set to an operational reception state. The detection device 121 comprises a detection sensor 128, for example, in the form of a GMR sensor, which is connected to a transistor T2 and is used in conjunction with the transistor T2 to generate a sensor signal.

By means of the detection sensor 128 a signal of an external signal source 2 may be detected. The external signal source 2 may, for example, have the form of a permanent magnet, an electromagnet or a so-called TMP magnet. The presence of a magnetic field M of the external signal source 2 may be detected by means of the detection sensor 128 in order to generate, using the transistor T2, a signal which depends on whether a magnetic field M is present or not and to feed said signal to the inversion circuit 123.

The inversion circuit 123 comprises a transistor T3, resistors R1, R2 and a capacitor C1 connected to one another to invert a signal received from the detection device 121.

If a magnetic field M is detected during the pulse duration A of a signal P1, by means of which the detection device 121 is transferred from an off state to the reception state, the detection device 121 generates a signal P2′ which has a low signal level (low signal). If, on the other hand, no magnetic field M is detected during the pulse duration A, the detection device 121 generates a signal P2 which has a high signal level (high signal). The respective signal is fed to the transistor T3 of the inversion circuit 123 and is converted to an inverted signal P3, P3′ by the inversion circuit 123. A low signal level P2′ is thus converted to a high signal level P3′ (High). In contrast, a high signal level P2 is converted to a low signal level P3 (Low).

The inverted signal is fed to an input 127 of the first timer circuit 120. The input 127 may, for example, be a so-called interrupt connection, which causes an interruption of the time routine of the first timer circuit 120 for the periodic switching of the functional device 10 and triggers an actuation of the switching device 124 via transistor T1 and thus a switching of the functional device 10 to the functional state, if a high signal level P3′ (High) is present at the input 127.

While a synchronous, periodic switching of the sensor device 10 is thus effected via the first timer circuit 120 using the oscillator circuit 125, the functional device 10 may be switched on asynchronously via the second timer circuit 122 and the detection device 121 in the presence of an external signal source 2. Using the second timer circuit 122 and the detection device 121, the system of the medical device 1 may thus be switched on as desired by a user if there is an acute need to perform a function, for example, to perform a measurement.

Both the first timer circuit 120 and the second timer circuit 122 are connected to a bus system 16, for example, a so-called SPI bus, via which the first timer circuit 120 and the second timer circuit 122 may, for example, be programmed.

The external signal source 2 may, for example, be a permanent magnet, an electromagnet or a so-called TMP magnet. To trigger a switching-on of the medical device 1, the external signal source 2 outside of the patient is brought into proximity with the medical device 1 (which is implanted in the patient), so that the magnetic field M of the external signal source 2 may be detected by the detection sensor 128 of the detection device 121.

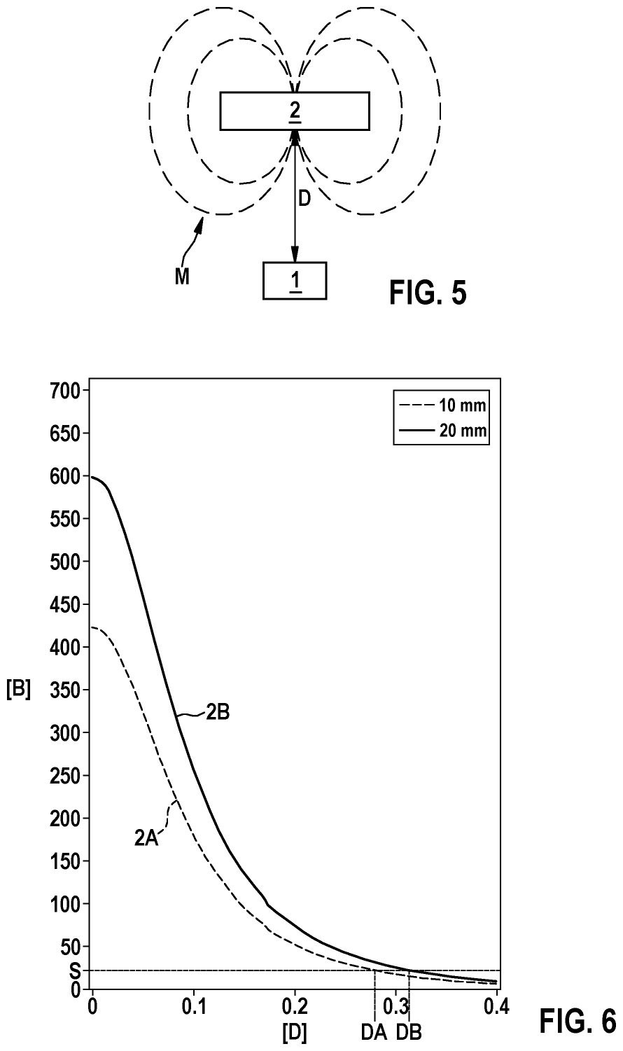

FIG. 5 shows the external signal source 2 at a spatial distance D to the medical device 1. FIG. 6 graphically shows the magnetic flux density B at the location of the medical device 1 in dependence of the distance D between the signal source 2 and the medical device 1, wherein the detection device 121 generates a signal indicating the presence of an external signal is source 2, for example, when the magnetic flux density B at the location of the medical device 1 exceeds a threshold S (when approaching the external signal source 2 towards the medical device 1, i.e., when reducing the distance D).

In FIG. 5, the magnetic flux density B is plotted over the distance D for different sizes of signal sources 2A, 2B and thus for magnetic fields M of different strengths. The threshold S is exceeded for signal source 2A at distance DA, and for signal source 2B at a (slightly larger) distance DB.

The presence of a magnetic field M is detected via the detection sensor 128 of the detection device 121, which is switched on via the second timer circuit 122 with a comparatively narrow clocking, for example, every 4 seconds, for a pulse duration A of, for example, 200 is. It hence may be detected with a comparatively narrow clocking whether a user approaches a signal source 2 towards the medical device 1 for switching on the functional device 10.

The medical device 1, in particular the functional device 10, is active only if it is switched on by the wake-up device 12. Once the functional device 10 has been switched on, it remains in its operational state for a predetermined period of time and then automatically reverts back to a (substantially) current-less, switched-off state until it is switched on again by the wake-up device 12.

In addition, the detection device 121 of the wake-up device 12 is not always active, but only if it is switched on by the second timer circuit 122.

Hence, substantially only the first timer circuit 120 and the second timer circuit 122 operate in a continuous fashion. The first timer circuit 120 and the second timer circuit 122 may each be realized, for example, by their own electronic chip, wherein each chip may be implemented, for example, in the sub-threshold technology and thus each timer circuit 120, 122 may exhibit a low energy consumption.

This is illustrated in FIG. 7.

Using pulses P1, the detection device 121 is periodically switched on. Each pulse P1 has a pulse duration A of, for example, 200 μs seconds. The pulses P1 are generated periodically by the second timer circuit 122 at a regular time interval B of, for example, 4 seconds.

In a state in which only the timer circuits 120, 122 are active, i.e., between two pulses P1, the system of the medical device 1 draws a current I1. During a pulse P1, i.e., in a switched-on phase of the detection device 121, the current rises to a value 13.

Between times Z1, Z2, the first timer circuit 120 is (periodically) calibrated using the calibration oscillator 126. In a phase between times Z1, Z2, the current thus increases to a value 12 in phases between two pulses P1 and to a value 14 during a pulse P1.

The idea(s) underlying the present invention is not limited to the embodiments described above, but may be implemented in other ways.

A medical device of the type described herein may be used, for example, as a measuring device for measuring a parameter within a patient, such as blood pressure, temperature, flow or the like. A medical device may alternatively or in addition have a therapy function, for example, a pacemaker function or a neurostimulation function.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device
10 Functional device
11 Memory device (RAM)
12 Wake-up device
120 First timer circuit
121 Detection device
122 Second timer circuit
123 Inversion circuit
124 Switching device
125 Oscillator circuit (RC oscillator)
126 Calibration oscillator (quartz oscillator)
127 Input
128 Detection sensor
13 Energy storage device
14 Sensor device
15 Housing
16 Bus system
17 Communication device
2 Signal source
3 External device
A Pulse duration
B Pulse interval
C1 Capacitor
D, DA, DB Distance
I1, I2, I3 Current
M Magnetic field
P1 Signal
P2, P2′ Signal
P3, P3′ Signal
R1, R2 Resistor
S Threshold
T1, T2, T3 Transistor
Z1, Z2 Time

1. An implantable medical device, comprising: an electronic functional device for performing a function of said implantable medical device, said functional device having an operational state for performing said function and a switched-off state, and a wake-up device for transferring said functional device from said switched-off state to said operational state, wherein the wake-up device comprises a first timer circuit for repeatedly transferring the functional device into the operational state according to a first timing scheme, a detection device for detecting a signal from a signal source external to the implantable medical device, and a second timer circuit for repeatedly transferring the detection device into a reception state according to a second timing scheme.

2. The implantable medical device according to claim 1, wherein the first timer circuit is configured to periodically transfer the functional device into the operational state.

3. The implantable medical device according to claim 1, wherein first timer circuit comprises an oscillator circuit adapted to generate a clock signal for determining a point in time to transfer the functional device to the operational state.

4. The implantable medical device according to claim 3, wherein the oscillator circuit comprises an RC oscillator.

5. The implantable medical device according to claim 3, wherein the first timer circuit comprises a calibration oscillator for calibrating the oscillator circuit.

6. The implantable medical device according to claim 5, wherein the first timer circuit is configured to repeatedly calibrate the oscillator circuit using the calibration oscillator according to a calibration time scheme.

7. The implantable medical device according to claim 5, wherein the calibration oscillator is formed by a quartz oscillator.

8. The implantable medical device according to claim 1, wherein the second timer circuit is configured to periodically switch the detection device to the reception state for a predetermined reception period.

9. The implantable medical device according to claim 1, wherein the detection device comprises a detection sensor detecting a signal from the signal source.

10. The implantable medical device according to claim 9, wherein the detection sensor formed by a GMR sensor.

11. The implantable medical device according to claim 1, wherein the detection device is configured to generate a signal which depends on the detection of a signal of the signal source.

12. The implantable medical device according to claim 11, wherein the wake-up device comprises an inversion circuit for inverting the signal generated by the detection device and for outputting an inverted signal obtained by the inversion.

13. The implantable medical device according to claim 12, wherein the inversion circuit is connected to the first timer circuit to output the inverted signal to the first timer circuit.

14. The implantable medical device according to claim 1, wherein the functional device, after being transferred to the operational state, remains in the operational state for a predetermined period of time for carrying out said function and then reverts to the switched-off state.

15. The implantable medical device according to claim 1, wherein the first timer circuit and/or the second timer circuit comprise at least one electronic component implemented in sub-threshold technology.
